SEQUENCE LISTING (4 sequences)

SEQ ID NO: 1 - DNA, 33 bp, artificial sequence
  Primer SV40TAG5′-BII′
ggcgagatctaccatggataaagttttaaacag

SEQ ID NO: 2 - DNA, 30 bp, artificial sequence
  Primer SV40TAG3′-XI′
ggcgctcgagttatgtttcaggttcagggg

SEQ ID NO: 3 - DNA, 55 bp, artificial sequence
  Feline herpesvirus polyA signal
caataaacatagcatacgttatgacatggtctaccgcgtcttatatggggacgac

SEQ ID NO: 4 - protein, 1346 aa, Artificial Sequence
  cTERT amino acid
MERGAQPGVGVRRLRNVAREEPFAAVLGALRGCYAEATPLEAFVRRLQEGGTGEVEVLRGDDAQCYRTFVSQCVVCVPRGARAIPRPICFQQLSSQSEVITRIVQRLCEKKKKNILAYGYSLLDENSCHFRVLPSSCIYSYLSNTVTETIRISGLWEILLSRIGDDVMMYLLEHCALFMLVPPSNCYQVCGQPIYELISRNVGPSPGFVRRRYSRFKHNSLLDYVRKRLVFHRHYLSKSQWWKCRPRRRGRVSSRRKRRSHRIQSLRSGYQPSAKVNFQAGRQISTVTARLEKQSCSSLCLPARAPSLKRKRDGEQVEITAKRVKIMEKEIEEQACSIVPDVNQSSSQRHGTSWHVAPRAVGLIKEHYISERSNSEMSGPSVVHRSHPGKRPVADKSSFPQGVQGNKRIKTGAEKRAESNRRGIEMYINPIHKPNRRGIERRINPTHKPELNSVQTEPMEGASSGDRKQENPPAHLAKQLPNTLSRSTVYFEKKFLLYSRSYQEYFPKSFILSRLQGCQAGGRRLIETIFLSQNPLKEQQNQSLPQQKWRKKRLPKRYWQMREIFQKLVKNHEKCPYLVFLRKNCPVLLSEACLKKTELTLQAALPGEAKVHKHTEHGKESTEGTAPNSFLAPPSVLACGQPERGEQHPAEGSDPLLRELLRQHSSHWQVYGFVRECLERVIPAELWGSSHNKCRFFKNVKAFISMGKYAKLSLQQLMWKMRVNDCVWLRLAKGNHSVPAYEHCYREEILAKFLYWLMDSYVIELLKSFFYITETMFQKNMLFYYRKFIWGKLQNIGIRDHFAKVHLRALSSEEMEVIRQKKYFPIASRLRFIPKMNGLRPVVRLSRVVEGQKLSKESREKKIQRYNTQLKNLFSVLNYERTVNTSIIGSSVFGRDDIYRKWKEFVTKVFESGGEMPHFYFVKGDVSRAFDTIPHKKLVEVISQVLKPESQTVYGIRWYAVIMITPTGKARKLYKRHVSTFEDFIPDMKQFVSKLQERTSLRNAIVVEQCLTFNENSSTLFTFFLQMLHNNILEIGHRYYIQCSGIPQGSILSTLLCSLCYGDMENKLLCGIQKDGVLIRLIDDFLLVTPHLMQARTFLRTIAAGIPEYGFLINAKKTVVNFPVDDIPGCSKFKHLPDCRLISWCGLLLDVQTLEVYCDYSSYAFTSIRSSLSFNSSRIAGKNMKCKLTAVLKLKCHPLLLDLKINSLQTVLINIYKIFLLQAYRFHACVLQLPFNQKVRNNPDFFLRIISDTASCCYFILKAKNPGVSLGSKDASGMFPFEAAEWLCYHAFIVKLSNHKVIYKCLLKPLKVYKMHLFGKIPRDTMELLKTVTEPSLCQDFKTILD

(19) United States
(12) Patent Application Publication
(10) Pub. No.: US 2019/0144831 A1
(43) Pub. Date: May 16, 2019

(54) IMMORTALISED CHICKEN EMBRYONIC EPITHELIAL KIDNEY CELLS

(71) Applicant: Intervet Inc., Madison, NJ (US)

(72) Inventor: Jaap Kool, Munster (DE)

(73) Assignee: Intervet Inc., Madison, NJ (US)

(21) Appl. No.: 16/060,252

(22) PCT Filed: Dec. 9, 2016
(86) PCT No.: PCT/EP2016/080446

§ 371 Date: Jun. 7, 2018

(30) Foreign Application Priority Data
Dec. 11, 2015 (EP) 15199511.5

(51) Int. Cl. C12N 5/071; C07K 14/005; C12N 9/12; A61K 39/145

(52) U.S. Cl. CPC C12N 5/0686; C07K 14/005; C12N 9/1276; C12Y 207/07049; C12N 2710/22062; C12N 2510/02; C12N 2510/04; C12N 2710/24252; A61K 39/145

(57) ABSTRACT

The present invention relates to immortalised chicken embryonic epithelial kidney cells, to cell cultures comprising such immortalised cells, to vaccines comprising such cells, to methods for the replication of avian viruses on such cells, and to methods for the preparation of such cells and such vaccines.

The present invention relates to immortalised chicken embryonic epithelial kidney cells, to cell cultures comprising such immortalised cells, to vaccines comprising such cells, to methods for the replication of avian viruses on such cells, and to methods for the preparation of such cells and such vaccines.

The propagation of viruses for the purpose of vaccine production requires the availability of susceptible host cells. Usually, depending on the virus species and the type of host cell used, these host cells will be grown in cell culture. For the propagation of many avian virus species there is the additional possibility of propagation in embryonated eggs. However in practice, many avian virus species are grown on primary chicken embryo fibroblast (CEF) cells. (Cells that are cultured directly from an animal are known as primary cells). Such primary CEF cells are susceptible to many different virus species and such viruses can often be grown to high titers in these cells.

In spite of the fact that CEF cells seem to be a good substrate for several avian viruses, it seems likely that in the egg the chorio-allantoic membrane is the main site for virus replication. This membrane is an epithelial cell layer.

For that reason, it is desirable to have an immortalized epithelial cell line available for growing avian viruses. More specifically it is important to have an immortalized epithelial cell line of kidney origin available.

One way of obtaining such cells is to isolate and grow primary Chicken Embryonic Epithelial Kidney cells and to try and wait for a spontaneous immortalization event to happen. However, for all avian cells, spontaneous immortalisation is very rare.

As a consequence, only a very few spontaneously immortalised chicken embryo cell lines have been reported at all.

Only three spontaneously immortalised chicken embryo fibroblast cell lines have been reported; DF-1 (U.S. Pat. No. 5,672,485), SC-1 and SC-2 (Christman, S. A. et al., Dissertation Abstracts Int. 65: 4414 (2004) (ISBN 0-496-06882-2).

Only one spontaneously immortalised chicken liver cell is described by Jeongyoon Lee et al., Poultry science 92: 1604-1612 (2013).

Also only one immortalized avian kidney epithelial cell has been described (Katsuyuki Kadoi, New Microbiologica 33: 393-397 (2010)). This immortalized cell is also a spontaneously immortalised cell.

Apart from the fact that finding and isolating a spontaneously immortalized chicken cell can be very time consuming, it also turns out that such immortalised chicken cells may show quite different characteristics such as variable growth rates and p53 levels over time, depending on the number of passages that they have had (Christman, S. A. et al., FEBS letters 579: 6705-6715 (2005)). However, especially for vaccine production purposes, cell lines are needed of which the characteristics remain the same over time, regardless the number of passages. The FDA has indicated that the development of minimally purified live attenuated viral vaccines in neoplastic cells that have been transformed by unknown mechanisms is discouraged (FDA; CBER Discussion on cell substrates May 12, 2000). Thus, spontaneously immortalised chicken cells are not considered really suitable as a source of cells for virus propagation for vaccine manufacturing purposes.

There are also methods known in the art for intentional immortalisation of cells. The advantage of such methods is that the coincidence factor, known to be a severe hindrance when aiming at spontaneously immortalised cells, is eliminated. For non-avian cells, especially mammalian cells, a frequently used method to obtain immortalised cells relies upon infection of primary cells with retroviruses or retroviral vectors, or transfection of primary cells with DNA molecules that comprise retroviruses or at least retroviral Long Terminal Repeat (LTR) sequences and sequences encoding DNA tumor virus oncoproteins such as Simian Virus SV40 T and t.

The SV40 T and t play a role in the inactivation of the retinoblastoma (Rb) and p53 proteins. A review paper by Deepika Ahuja et al., about SV40 T encoding large T and t provides insight in the mechanisms of action of these proteins (Oncogene 24: 7729-7745 (2005)). Basically, the SV40 T gene products T and t inhibit the p53 and Rb-family of tumor suppressors.

LTRs are retroviral elements that comprise all required signals for retroviral gene expression: enhancer, promoter, transcription initiation, transcription terminator and polyadenylation signal.

However, these LTRs are suspected of having tumorigenic effects. This is due to the fact that they are known to cis-activate other cellular genes and the fact that they may recombine with other retroviral sequences in the cellular genome (Mosier, D. E., Applied Biosafety 9: 68-75 (2004)). Thus, a severe disadvantage of the use of retroviral DNA comprising LTRs or at least long retroviral sequences for transfecting cells is that in such cases LTR sequences will be introduced into the DNA of the immortalised cells.

For avian cells, only a few example of a successful deliberate immortalisation are known.

Only one example of successful immortalization with a DNA molecule comprising LTRs and expressing SV40 T antigen is known: such deliberately immortalised chicken embryo fibroblast cells are described in PCT Application WO 97/44443.

A different approach was followed in U.S. Pat. No. 6,255,108, where an anti-apoptotic gene, the bcl-2 gene was introduced in the cell, preferably followed by the introduction of SV40 T and t. This approach however also used LTR sequences in order to obtain stable integration of the bcl-2 gene.

The use of the 12S adenoviral E1A protein for the immortalization of avian cells, again using LTR-sequences for stable integration of the adenoviral gene is described by Guilhot C. et al., in Oncogene 8: 619-624 (1993).

In WO2005/042728, the approach using SV40 T and t antigen was considered to be too aggressive to silence RB and P53 in avian cells. In this patent application, it is described how two different genes, one interfering with RB and one interfering with P53 were used to silence the RB/P53 activity.

A disadvantage of this approach is that it relies on the stable integration of not just one, but two genes in order to obtain immortalisation. And the loss of one of the two genes will lead to cell death.

No deliberately immortalised chicken embryonic epithelial kidney cells have been described in the art up till now.

Apparently, immortalization of chicken cells with a DNA molecule comprising LTRs and expressing SV40 T antigen is not very successful. Soo-Hyun Kim described transfection of CEF cells with a retrovirus encoding SV40 T antigen, in an attempt to immortalise CEF cells through inactivation of the retinoblastoma (Rb) and p53 tumor suppressors. (J. Cell Science 119: 2435-2443 (2006)). This however only led to a slight extension of the life span of the CEF cells, not to immortalisation.

Kim at that time suggested that a possible explanation for the limited lifespan extension could be the continued erosion of telomeres. However, the situation is complicated by the fact that chicken cells contain both macro- and micro-chromosomes with different size classes of telomeric repeats, some of which are interstitial. Thus, at this juncture the dynamics of telomere erosion and repair and their effect on immortalisation of chicken cells remain unclear.

This touches upon the basic question whether a critical role in immortalisation of CEF relates to the loss of p53 and Rb or the activation of telomerase (Campisi, J., Exp. Gerontol. 36: 607-618 (2001) and Sherr, J. C. and DePinho, R. A., Cell 102: 407-410 (2000)).

It can be derived from recent experiments that, contrary to Kim's suggestion, the role of telomerase in the immortalisation of CEF cells appears not to be a critical one. First of all, it is striking that in the spontaneously immortalised CEF cell line SC-1 (see above) no telomerase expression is detectable (Christman, S. A. et al., FEBS letters 579: 6705-6715 (2005)).

Secondly, in several straightforward experiments in which chicken cells were transduced or transfected with cTR, cTERT or both cTR and cTERT, no immortalised chicken cells were obtained (Swanberg, S. E. et al., Exp. Gerontol. 45: 647-654 (2010)).

It has now contrary to all expectations been found that stably transfected CEEK cell lines can be obtained through transfection of CEEK cells, without the use of LTR sequences, with a DNA molecule comprising transposon inverted repeats for the integration of the DNA molecule into the cellular genome and a combination of both the gene encoding the SV40 T and t antigen or at least T under the control of a suitable promoter, and the gene encoding chicken telomerase (cTERT) under the control of a suitable promoter. The transposon inverted repeats play a role in the stable integration of the gene encoding the SV40 T and t antigen or at least T and the gene encoding (cTERT) into the genome of the CEEK cell, which is a prerequisite for obtaining a stably transfected immortalised CEEK cell according to the invention.

For the purpose of the present invention, an immortalized cell line is a population of cells (in this case Chicken Embryonic Epithelial Kidney (CEEK) cells) originating from a multicellular organism, which would normally not proliferate indefinitely but, due to mutation, has evaded normal cellular senescence and instead can keep undergoing cell division. Such cells have escaped the normal limitation of growth for only a finite number of division cycles.

Thus, a first embodiment of the present invention relates to a stably transfected immortalised chicken embryonic epithelial kidney (CEEK) cell, characterized in that the stably transfected immortalised CEEK cell comprises a gene encoding the SV40 T antigen under the control of a suitable promoter, and comprises a gene encoding chicken telomerase; cTERT under the control of a suitable promoter and does not comprise exogenous retroviral Long Terminal Repeat DNA.

Exogenous retroviral LTR DNA is considered to be DNA that is brought into a chicken embryonic epithelial kidney cell during the process of immortalisation as described above.

Such CEEK cells according to the invention thus express at least the SV40 T antigen, or the SV40 T antigen and t antigen, and they express chicken telomerase. In this last respect, the expression of chicken telomerase, the CEEK cells according to the invention differ from natural embryonic epithelial kidney cells in that they express chicken telomerase under the control of an exogenous promoter, or at least under the control of a heterologous promoter, i.e. not the promoter that is driving chicken telomerase expression in vivo.

The details and characteristics of such immortalised CEEK cells as well as methods for the preparation of such CEEK cells are extensively described below.

A second embodiment of the present invention relates to methods for the preparation of such immortalised CEEK cell lines.

Methods for the preparation of an immortalised CEEK cell line according to the invention basically comprise the following steps:

a) the step of obtaining primary CEEK cells. This step is explained in detail in the Examples section.

b) the step of transfecting said CEEK cells with 1) a DNA molecule free of LTR sequences, comprising transposon inverted repeats and comprising a gene encoding the SV40 T antigen under the control of a suitable promoter, 2) a DNA molecule free of LTR sequences, comprising transposon inverted repeats and comprising a gene encoding chicken telomerase; cTERT under the control of a suitable promoter and 3) a DNA molecule comprising a gene encoding transposase under the control of a suitable promoter.

The DNA molecule comprising a gene encoding transposase under the control of a suitable promoter needs not necessarily to be free of LTR sequences, because this DNA molecule itself does not comprise transposon sequences and will thus not likely be integrated in the host's genome. Nevertheless, in order to avoid unintended accidental integration, preferably the DNA molecule comprising a gene encoding transposase under the control of a suitable promoter is free of LTR sequences.

For reasons of efficiency, in practice the transfection would preferably be done with a single DNA molecule free of LTR sequences, comprising transposon inverted repeats, comprising both a gene encoding the SV40 T antigen under the control of a suitable promoter and a gene encoding chicken telomerase under the control of a suitable promoter and comprising a gene encoding transposase under the control of a suitable promoter.

The transposase activity is necessary only during the first steps of the immortalisation process for integration of the DNA in the CEEK cell genome. Once the DNA(s) is/are integrated, the transposase is no longer needed. Afterwards it may even become detrimental to the stability of the cells.

Thus, more preferably the step of transfecting said CEEK cells would be done with 1) a single DNA molecule free of LTR sequences, comprising transposon inverted repeats and comprising both a gene encoding the SV40 T antigen under the control of a suitable promoter and a gene encoding chicken telomerase under the control of a suitable promoter and 2) a DNA molecule, preferably free of LTR sequences, only comprising the transposase-gene under the control of a suitable promoter without transposon sequences.

Transfection can be done in many ways known in the art. Commercial kits for transfection are currently available through i.a. Bio-Rad (Life Science (Research, Education, Process Separations, Food Science), Life Science Research, 2000 Alfred Nobel Drive, Hercules, Calif. 94547, USA) and Invitrogen (Life Technology, 3175 Staley Road, Grand Island, N.Y. 14072, USA). Commonly used reagent-based transfection methods comprise the use of lipids, calcium phosphate, cationic polymers, DEAE-dextran, activated dendrimers and magnetic beads. Instrument-based methods comprise electroporation, nucleofection and micro-injection.

A DNA molecule free of LTR sequences, comprising transposon inverted repeats and comprising a gene encoding the SV40 T antigen under the control of a suitable promoter and/or the gene encoding chicken telomerase under the control of a suitable promoter could e.g. be a plasmid. The plasmid may be in a circular or linear form when it is used for the transfection step.

The use of transposons as such is well-known in the art. A paper by Ivics, Z. and Izsvak Z. extensively reviews transposons and their use, and provides insight in the mechanisms of action of transposons (Mobile DNA 1: 25-39 (2010)). Transposons can be viewed as natural DNA transfer vehicles that, similar to integrating viruses, are capable of efficient genomic insertion, mediated by transposase.

In principle, the transposons remain stably present in the cellular genome after integration in the genome. Therefore, preferably immortalized CEEK cells according to the invention comprise transposon sequences such as the transposon inverted repeats.

A large number of suitable promoters for the expression of the SV40 T antigen and the cTERT are known in the art, which are recognized for their efficient level of expression. It is known that promoters which are transcriptionally active in mammalian cells also function well in avian cells. Such promoters include classic promoters such as the (human) cytomegalovirus immediate early promoter (Sun-Young Lee et al., Journal of Biomedical Science 6: 8-17 (1999), Seed, B. et al., Nature 329, 840-842, 1987; Fynan, E. F. et al., PNAS 90, 11478-11482, 1993; Ulmer, J. B. et al., Science 259, 1745-1748, 1993), the Human Cytomegalovirus enhancer-promoter (Donofrio G., et al., Clinical and Vaccine Immunology 13: 1246-1254, (2006)), the Mouse Cytomegalovirus immediate early (MCMVie1) promoter, the Mouse Cytomegalovirus early (MCMVe1) promoter, SV40 immediate early promoter (Sprague J. et al., J. Virology 45, 773, 1983), the SV-40 promoter (Berman, P. W. et al., Science, 222, 524-527, 1983), the metallothionein promoter (Brinster, R. L. et al., Nature 296, 39-42, 1982), the heat shock promoter (Voellmy et al., Proc. Natl. Acad. Sci. USA, 82, 4949-53, 1985), the major late promoter of Ad2 and the β-actin promoter (Tang et al., Nature 356, 152-154, 1992).

A preferred promoter is the CAG promoter. (Miyazaki, J; Takaki, S; Araki, K; Tashiro, F; Tominaga, A; Takatsu, K; Yamamura, K., Gene 79 (2): 269-277 (1989), and Niwa, H; Yamamura, K; Miyazaki, J., Gene 108 (2): 193-199 (1991)).

c) the step of selecting cells that are capable of sustained proliferation.

CEEK cells that are capable of sustained proliferation are cells that keep proliferating for at least 25 population doublings. The cell cycle, or cell-division cycle, is the series of events that take place in a cell leading to its division and duplication (the cell replication). The selection of cells that are capable of sustained proliferation is a very simple process for the following reason: primary CEEK cells are, even in the most optimal situation, not capable of dividing outside their natural environment, the avian embryo, for more than about 15 times. After an initial phase of proliferation, the proliferation rate of live primary CEEK cells after isolation decreases over time and eventually all primary CEEK cells enter into a non-proliferative stage. As a consequence they will die off after a maximum of about 15 population doublings.

This means that if there is an increase in the number of cells after about 15 population doublings, especially after about 25 population doublings, this must be due to the fact that one or more cells have successfully been transfected and that the gene encoding the SV40 T antigen and the gene encoding cTERT are inserted in the cellular genome. So basically the process is self-selecting: maintenance of CEEK cells some of which were successfully transfected, in a suitable cell growth medium will automatically lead to replication of these transfected cells, whereas non-immortalised cells will stop dividing and die off. Suitable cell growth media are known in the art (see Kim, 2006 and Hernandez, 2010 above) and are described i.a. in the Examples section.

Further guidance about cell culture conditions can be found in the Examples.

As said above, usually, cells are selected that have been cultured for at least 25 cell cycles. For such cells it can reasonably be assumed that they are successfully immortalized CEEK cells, since primary CEEK cells will usually not replicate more than about 15 times in vitro after isolation from the chicken embryo.

Thus, one embodiment of the present invention relates to a method for the preparation of an immortalised CEEK cell according to the invention, wherein that said method comprises the steps of
a) obtaining primary CEEK cells,
b) transfecting said CEEK cells with 1) a DNA molecule free of LTR sequences, comprising transposon inverted repeats and comprising a gene encoding the SV40 T antigen under the control of a suitable promoter, 2) a DNA molecule free of LTR sequences, comprising transposon inverted repeats and comprising a gene encoding chicken telomerase; cTERT under the control of a suitable promoter and 3) a DNA molecule comprising a gene encoding transposase under the control of a suitable promoter.
c) selecting CEEK cells that have been cultured for at least 25 cell cycles.

A preferred form of this embodiment relates to a method for the preparation of an immortalised CEEK cell according to the invention, characterized in that said method comprises the steps of
a) obtaining primary CEEK cells,
b) transfecting said primary CEEK cells with a single DNA molecule free of LTR sequences, comprising transposon inverted repeats, comprising both a gene encoding the SV40 T antigen under the control of a suitable promoter and a gene encoding chicken telomerase under the control of a suitable promoter and comprising a gene encoding transposase under the control of a suitable promoter,
c) selecting CEEK cells that have been cultured for at least 25 cell cycles.

A more preferred form of this embodiment relates to a method for the preparation of an immortalised CEEK cell according to the invention, characterized in that said method comprises the steps of
a) obtaining primary CEEK cells,
b) transfecting said primary CEEK cells with 1) a DNA molecule free of LTR sequences, comprising transposon inverted repeats, comprising both a gene encoding the SV40 T antigen under the control of a suitable promoter and a gene encoding chicken telomerase under the control of a suitable promoter and with 2) a DNA molecule free of LTR sequences, comprising a gene encoding transposase under the control of a suitable promoter,
c) selecting CEEK cells that have been cultured for at least 25 cell cycles.

In exceptional cases, cells that went through around 25 cell cycles may still show instable behavior, e.g. due to the fact that the transposon has integrated in the cellular genome at a very critical site, or due to instable integration of the gene encoding the SV40 T antigen or encoding cTERT. Therefore, in practice preferably cells are selected that have been cultured for at least 40, 50, 60, 70, 80, 90, 100, 110, 120, 130, 140, 150 or even 160 cell cycles, in that order of preference. The chances of any instability becoming manifest do decrease with the amount of cell cycles of the selected immortalised CEEK cells.

Thus, preferably, cells are selected that have been cultured for at least 40, 50, 60, 70, 80, 90, 100, 110, 120, 130, 140, 150 or even 160 cell cycles, in that order of preference.

A third embodiment of the present invention relates to methods for the replication of an avian virus or an avian viral vector, said methods comprising the steps of

a) culturing an immortalised CEEK cells according to the invention,

b) contacting the immortalised CEEK cells with the avian virus or avian viral vector and

c) allowing the avian virus or avian viral vector to replicate.

Avian viruses of special interest are the following avian viruses: Herpes virus of turkey (HVT), Marek's virus, Newcastle Disease virus (NDV), Infectious Bronchitis virus (IBV), Infectious Bursal Disease virus (IBDV), Egg Drop Syndrome virus (EDSV), Reovirus (RV) and Turkey Rhinotracheitis virus (TRT). For all these viruses, vaccines are known in the art, either on the basis of live attenuated viruses, inactivated viruses or recombinant viruses; viral vectors, expressing immunogenic components of any of these viruses.

A viral vector is a virus that carries an additional gene, not present in the wild-type form of the virus. Viral vectors are very well-known in the art. Viral vectors can be used to carry e.g. a foreign bacterial gene or a foreign viral gene. Usually, the additional gene is placed under the control of a suitable promoter. Examples of such viral vectors are e.g. a HVT vector comprising the IBDV VP2-gene, the IBV-spike protein gene, the avian influenza HA gene, the ILT gD/gI protein gene or the NDV F-gene.

Thus, a preferred form of this embodiment relates to methods of replicating an avian virus or an avian viral vector, according to the invention, wherein the avian virus or the avian viral vector is selected from the group of avian viruses consisting of Marek's Disease virus (MDV), the MDV-related Herpes virus of turkey (HVT), Newcastle Disease virus (NDV), Infectious Bronchitis virus (IBV), Infectious Bursal Disease virus (IBDV), Egg Drop Syndrome virus (EDSV), Reovirus (RV), Turkey Rhinotracheitis virus (TRT) and a HVT vector comprising the IBDV VP2-gene, the IBV-spike protein gene, the avian influenza HA gene, the ILT gD/gI protein gene or the NDV F-gene.

A fourth embodiment of the present invention relates to a cell culture comprising an immortalised CEEK cell according to the invention.

A preferred form of this embodiment relates to such a cell culture that is infected with an avian virus or an avian viral vector.

A more preferred form of this embodiment relates to such a cell culture that is infected with an avian virus or an avian viral vector selected from the group consisting of Marek's Disease virus (MDV), the MDV-related Herpes virus of turkey (HVT), Newcastle Disease virus (NDV), Infectious Bronchitis virus (IBV), Infectious Bursal Disease virus (IBDV), Egg Drop Syndrome virus (EDSV), Turkey Rhinotracheitis virus (TRT), Reovirus (RV) and a HVT vector comprising the IBDV VP2-gene, the IBV-spike protein gene, the avian influenza HA gene, the ILT gD/gI protein gene or the NDV F-gene.

A fifth embodiment of the present invention relates to methods for the preparation of a vaccine comprising an avian virus or an avian viral vector wherein that method comprises the step of mixing a cell culture according to the invention wherein the cell culture is infected with an avian virus or an avian viral vector, with a pharmaceutically acceptable carrier.

In a preferred form of this embodiment the avian virus or an avian viral vector is selected from the group consisting of Marek's Disease virus (MDV), the MDV-related Herpes virus of turkey (HVT), Newcastle Disease virus (NDV), Infectious Bronchitis virus (IBV), Infectious Bursal Disease virus (IBDV), Egg Drop Syndrome virus (EDSV), Reovirus (RV), Turkey Rhinotracheitis virus (TRT) and a HVT vector comprising the IBDV VP2-gene and/or the NDV F-gene.

A sixth embodiment of the present invention relates to methods for the preparation of a vaccine comprising an avian virus or an avian viral vector, wherein the method comprises the steps of

a) infecting a cell culture of CEEK cells according to the invention with an avian virus or an avian viral vector

b) replicating said avian virus or an avian viral vector

c) isolating the progeny virus

d) mixing the progeny virus with a pharmaceutically acceptable carrier

A preferred form of this embodiment relates to such a vaccine wherein the avian virus or an avian viral vector is selected from the group consisting of Marek's Disease virus (MDV), the MDV-related Herpes virus of turkey (HVT), Newcastle Disease virus (NDV), Infectious Bronchitis virus (IBV), Infectious Bursal Disease virus (IBDV), Egg Drop Syndrome virus (EDSV), Reovirus (RV), Turkey Rhinotracheitis virus (TRT) and a HVT vector comprising the IBDV VP2-gene and/or the NDV F-gene.

Finally, yet another embodiment relates to vaccines comprising a cell culture according to the invention and a pharmaceutically acceptable carrier.

### LEGEND TO THE FIGURES

FIG. 1: Vector maps for Plasmid #02 (pPB-CAG-ChTERT) (A) and Plasmid #03 (pPB-CAG-SV40 T Ag) (B).

FIG. 2: cTERT amino acid sequence. * indicates stop codon.

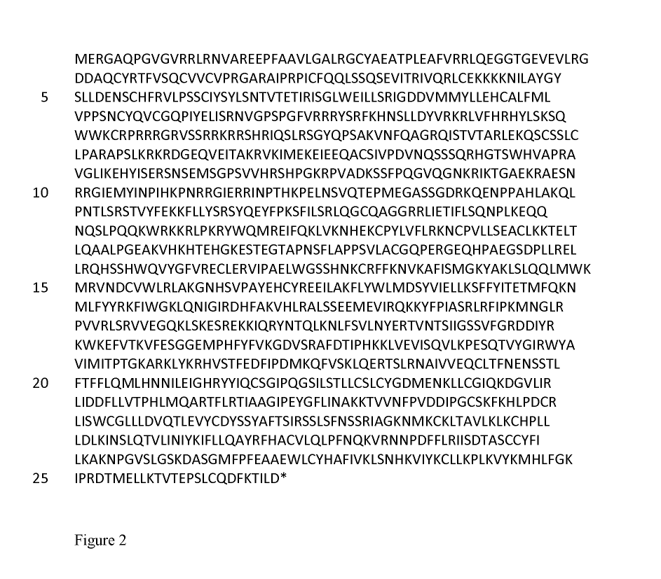

FIG. 3: indication of the location of the cells in the high density “F4” band of a Percoll gradient.

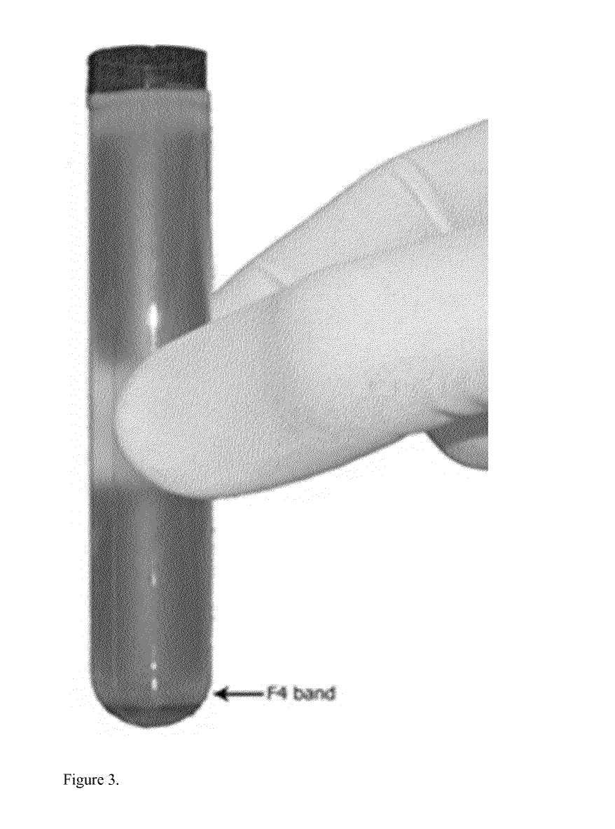

FIG. 4: Growth curve of the various transfected CEEK cells. It is indicated in the figure which plasmid(s) has/have been use for the transfection of the respective cells. Cell numbers were determined at each passage to calculate the population doublings per passage.

FIG. 5: This figure shows the epithelial morphology of primary CEEK cells that were transformed with Plasmid #03 (and Plasmid #04) in their 5^{th} passage, and of primary CEEK cells that were transformed with Plasmid #03 followed by a further transfection with Plasmid #02 (and Plasmid #04) after 17 passages, in their 25^{th} passage.

FIG. 6: This figure shows the epithelial morphology of primary CEEK cells that were transformed with Plasmid #03 (and Plasmid #04) in their 5^{th} passage, and of primary CEEK cells that were transformed with Plasmid #02 and Plasmid #03 (and Plasmid #04), in their 25^{th} passage.

### ABBREVIATIONS

Corning™ BioCoat™ Collagen I Rectangular Canted Neck Cell Culture Flask with Vented Cap

“Corning™ BioCoat™ Collagen I coated T25 culture flask #356484”

| | | | |
|---|---|---|---|
| 356484 | 50/Cs. | 70 mL | 25 cm₂ |
| 356485 | 50/Cs. | 250 mL | 75 cm₂ |
| 356486 | 40/Cs. | 600 mL | 150 cm₂ |
| 356487 | 40/Cs. | 750 mL | 175 cm₂ |

Corning™ BioCoat™ Collagen I Multiwell Plates

| | | |
|---|---|---|
| 356400 | 50/Cs. | 6 well |
| 356500 | 50/Cs. | 12 well |
| 356408 | 50/Cs. | 24 well |
| 356407 | 50/Cs. | 96 well |

Nomenclature of Cells:

CEK: Primary ex-vivo Chicken Embryonic Kidney cells

CEEK-TT1: Chicken Embryonic Epithelial Kidney cells—SV40 T Ag & ChTERT no. 1

CEEK-TT2: Chicken Embryonic Epithelial Kidney cells—SV40 T Ag & ChTERT no. 2

Nomenclature of the Plasmids Used:
Plasmid #01: pPB-CAG-EBNXN (Yusa et al., 2011): empty piggyBac transposon plasmid
Plasmid #02: pPB-CAG-ChTERT: piggyBac transposon plasmid containing Gallus gallus telomerase reverse transcriptase (ChTERT; # AAS75793.1|seq: 1-1346) followed by a feline herpesvirus polyA signal sequence (CAATAAACATAGCATACGTTATGACATGGTCTACCGCGTCTTA TATGGGGACGAC) (Willemse et al., 1995)
Plasmid #03: pPB-CAG-SV40 T Ag: piggyBac transposon plasmid containing Simian Virus 40 large T antigen (#EF579663.1|seq: 2619-5091)
Plasmid #02 & #03: Simultaneous transfection of plasmid #02 and #03 (CEEK-TT1)
Plasmid #03 p17 & #02: Initially transfection of plasmid #03, passaged 17 times, and subsequently transfection with plasmid #02 (CEEK-TT2)
Plasmid #04: pPB-CMV-hyPBase (Yusa et al., 2011), encodes for transposase

### EXAMPLES

### Example 1: Immortalization of Chicken Embryonic Epithelial Kidney Cells

Plasmids

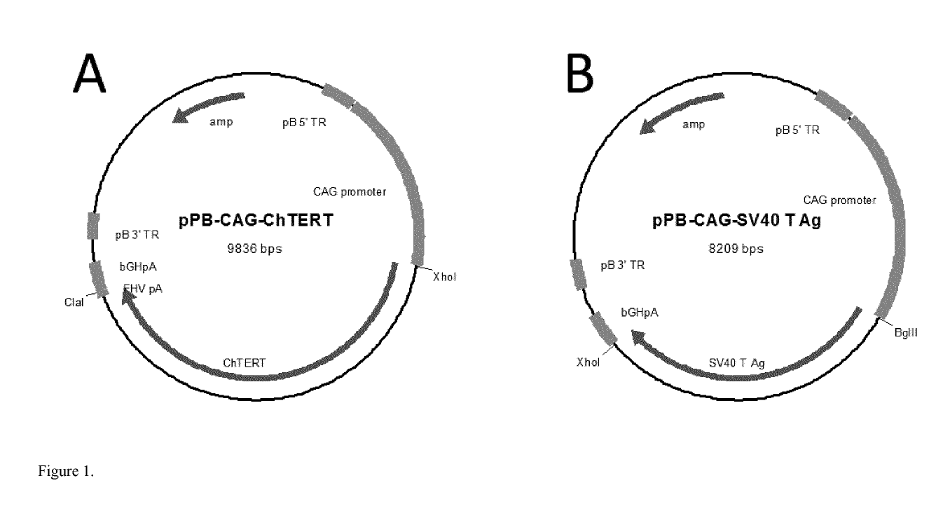

The sequence encoding ChTERT (Accession number #AAS75793.1) followed by a stop codon and a feline herpesvirus polyA signal sequence (CAATAAACATAGCATACGTTATGACATGGTCTACCGCGTCTTATATGGGGACGAC) (Willemse et al., 1995) was generated synthetically, sequenced and cloned into pPB-CAG-EBNXN (Yusa et al., 2009), henceforward called “plasmid #01”, subsequently using the XhoI-ClaI sites to create pPB-CAG-ChTERT, henceforward called “plasmid #02” (FIG. 1A). Plasmid DNA for transfection into CEEK cells was isolated using the Qiagen EndoFree plasmid maxi kit (Qiagen).

To construct pPB-CAG-SV40 T Ag, henceforward called “plasmid #03”, XhoI and BglII sites were added to SV40 T Ag by PCR using primers ‘W40 Tag 5′-BII’ (5′-GGCGAGATCTACCATGGATAAAGTTTTAAACAG-3′) and ‘W40 Tag 3′-XI’ (5′-GGCGCTCGAGTTATGTTTCAGGTTCAGGGG-3′). Phusion DNA polymerase was used for PCR according to the manufacturer's protocol (New England Biolabs). The fragment was cloned into pCR-Blunt (Life Technologies) and verified by sequencing. Next, SV40 T Ag was excised from pCR-Blunt and cloned into plasmid #01 using the BglII-XhoI sites to create plasmid #03 (FIG. 1B). The final construct was verified by sequencing.

Isolation, Purification and Growth of CEEK Cells.

Ten fertilized SPF eggs were incubated at 37° C. for nineteen days and used for isolation of primary chicken embryonic kidney cells. Embryos were harvested from the eggs under sterile conditions. The kidneys were removed and washed in sterile PBS and dissociated using a trypsin solution. After dissociation, fetal calf serum was added to inactivate trypsin. The isolated cells were centrifuged for 10 minutes at 600×g at 4° C. Pelleted cells were resuspended in 199/F10 medium containing N.P.P.T., stained for viability, and counted. 1.7*10⁶ cells/cm2 were plated in culture flasks and incubated at 37° C. and 5% CO₂.

The 199/F10 medium contains:
0.5× concentrated medium 199 (MP BIOMEDICALS #091020122)
0.5× concentrated HAM's F-10 nutrient mix (MP BIOMEDICALS #091040122)
1.48 gram/litre Tryptose phosphate broth (BECTON DICKINSON BV #260200)
1.10 gram/litre Sodium Bicarbonate (MERCK CHEMICALS BV #1063232500)
1.00 gram/litre HEPES (MERCK CHEMICALS BV #1101101000)

N.P.P.T. solution consists of 7.35 g/l Neomycin sulphate, 0.63 g/l Polymyxin B sulphate, 0.25 mg/l Pimafucin, 1.0 g/l Tylosin tartrate.

After 2 hours the non-attached cells were removed from the culture flask and centrifuged for 10 minutes at 400×g at 4° C. The pelleted cells were resuspended in 11 ml 1:1 Percoll (GE Healthcare)/2× concentrated Krebs-Henseleit buffer (Sigma) and centrifuged for 30 minutes at 17.500×g at 4° C. (Sutterlin, 1998). Cells in the high-density “F4” band (Sutterlin, 1998) were removed using a needle and syringe and then washed three times in HBSS-buffer (Sigma). The cells were stained for viability and counted before being seeded in 199/F10 medium containing: 2% FCS (Moregate), 1% Chicken serum (Sigma), 4.2 mg/litre Insulin (Sigma), 3.8 mg/litre Transferrin (Sigma), 5 μg/litre Selenite (Sigma), 2 mM L-Glutamin, N.P.P.T. solution, 1.1 g/l Sodium Benzyl Penicillin, 1.9 g/l Dihydrostreptomycin sulfate, “Renal Epithelial Cell Growth medium 2 Supplement Pack, without FCS” (Promocell C-39605). The cells were seeded at a density of 5.000-30.000 cells/cm2 in Corning™ BioCoat™ Collagen I coated culture flasks #356484-356487 and incubated at 40° C. and 5% CO₂. Supplement Pack was added to the culture medium during the first 3 passages of the cell culture, but was omitted from the medium from passage 4 onwards.

Transfection

Immediately after isolation and purification of primary CEEK cells 1.0*10⁶ viable cells were transfected in 100 μl Primary cell buffer P1+supplement (Lonza Cologne AG) using program CM-102 of the Nucleofector 4D device (Lonza Cologne AG). Cells were transfected with 1.6 μg plasmid #02, 1.6 μg plasmid #03 or with both plasmids (2*1.6 μg), and with 0.4 μg plasmid #04 or, as a control, with 1.6 μg plasmid #01 and 0.4 μg plasmid #04. After the pulse, cells were left at RT for 10 min. Next, 500 μl RPMI-1640 (40° C.) was slowly added to the cells and cells were incubated at 40° C. for 5 minutes. Then, cells were carefully resuspended, seeded in Corning™ BioCoat™ Collagen I coated T25 culture flasks #356484 in 199/F10 medium containing: 2% FCS (Moregate), 1% Chicken serum (Sigma), 4.2 mg/litre Insulin (Sigma), 3.8 mg/litre Transferrin (Sigma), 5 μg/litre Selenite (Sigma), 2 mM L-Glutamin, N.P.P.T. solution (7.35 g/l Neomycin sulphate, 0.63 g/l Polymyxin B sulphate, 0.25 mg/l Pimafucin, 1.0 g/l Tylosin tartrate), 1.1 g/l Sodium Benzyl Penicillin, 1.9 g/l Dihydrostreptomycin sulfate, “Renal Epithelial Cell Growth medium 2 Supplement Pack, without FCS” (Promocell C-39605) and incubated at 40° C. and 5% CO₂.

In addition, CEEK cells stably transfected with plasmid #03 were transfected after 17 passages with 1.6 μg Plasmid #02 (pPB-CAG-ChTERT) and 0.4 μg plasmid #04 using the same protocol (for a motivation, see under results; CEEK-TT2).

Tissue Culture

After transfection, cultures were grown in growth medium (199/F10 medium containing: 2% FCS (Moregate), 1% Chicken serum (Sigma), 4.2 mg/litre Insulin (Sigma), 3.8 mg/litre Transferrin (Sigma), 5 μg/litre Selenite (Sigma), 2 mM L-Glutamin, N.P.P.T. solution (7.35 g/l Neomycin sulphate, 0.63 g/l Polymyxin B sulphate, 0.25 mg/l Pimafucin, 1.0 g/l Tylosin tartrate), 1.1 g/l Sodium Benzyl Penicillin, 1.9 g/l Dihydrostreptomycin sulfate, “Renal Epithelial Cell Growth medium 2 Supplement Pack, without FCS” (Promocell C-39605) and routinely passaged upon 80-100% confluency. Supplement Pack was added to the culture medium during the first 3 passages of the cell culture, but was omitted from the medium from passage 4 onwards. After removal of the medium, cells were washed with PBS and detached using Accutase (Sigma) at 37° C. Cells were resuspended in growth medium and centrifuged for 5 minutes at 300×g RT. The pelleted cells were resuspended in growth medium and counted using a Bürker-Türk counting chamber. Cells were plated in fresh medium in Corning™ BioCoat™ Collagen I coated culture flasks #356484-356487 and incubated at 40° C. and 5% CO₂. Transfected CEEK cells were frozen for liquid nitrogen storage at different passages in standard medium containing 10% dimethylsulfoxide and 10% FCS. The number of population doublings was calculated using the following equation:

$$Population doublings = \frac{Log Nt - Log N}{Log 2}$$

where Nt was the number of viable cells at the end of the growth period and N the number of plated cells (Venkatesan and Price, 1998). Cells were photographed using an Olympus DP21 camera coupled to an Olympus CKX41 microscope.

Results

Immortalization of Primary CEEK Cells by Co-Expression of SV40 T Antigen and ChTERT.

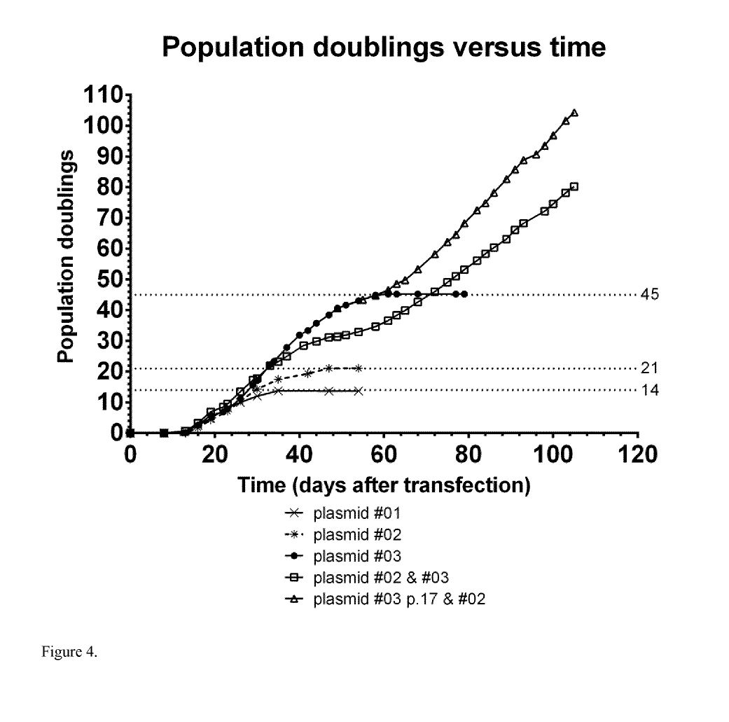

Primary CEEK cells transfected with plasmid #01 & #04 could be cultured for 10 passages, however the morphology of these cells quickly adapted a senescent morphology and cells proliferated slowly. After 14 population doublings the cells stopped proliferating and died. Primary CEEK cells transfected with plasmid #02 & #04 showed a comparable phenotype and stopped proliferating and died after 21 population doublings. In contrast, when transfected with plasmid #03 & #04, the cells proliferated well with a high proliferation rate and could be kept in culture for at least 45 population doublings. Although these plasmid #03 & #04 transfected cells clearly had an extended lifespan compared to the plasmid #01 & #04 and plasmid #02 & #04 transfected cells, they eventually stopped proliferating and died at the end of the extended lifespan, i.e. they went into ‘mitotic crisis’. Only primary CEEK cells stably transfected with plasmid #02 & #03 & #04 continued to proliferate indefinitely. These cells have been passaged at least 35 times by now and have performed at least 100 PDs (FIG. 4 growth curve). After 35 passages the cells are still proliferating vigorously and have an epithelial-like morphology Therefore, it can be concluded that they represent an immortalized cell line of chicken embryonic epithelial kidney cells. This cell line is referred to as CEEK-TT1 (Chicken Embryonic Epithelial Kidney cells—SV40 T Ag & ChTERT no. 1).

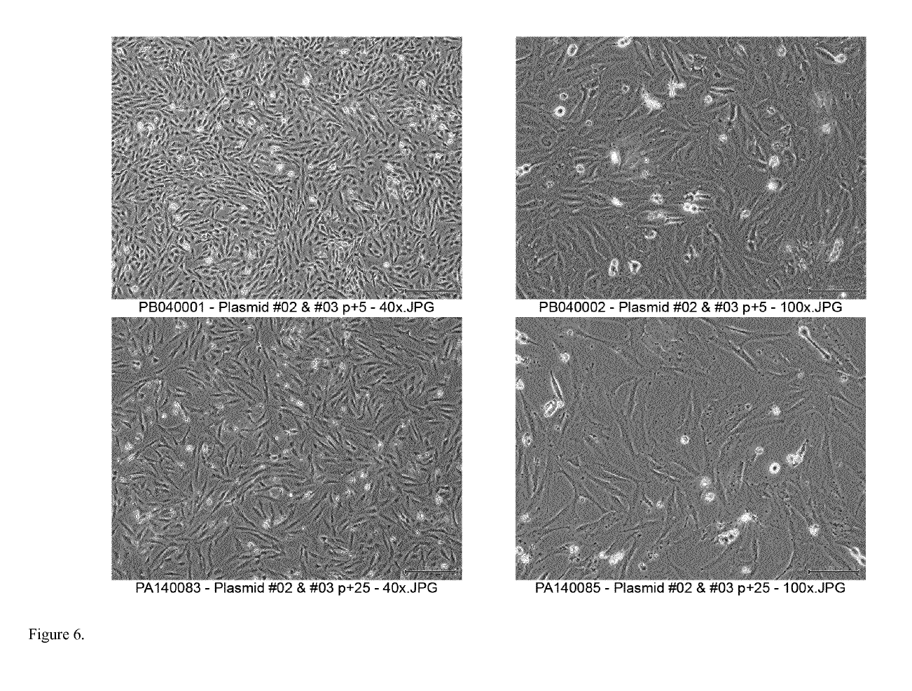

The cells have an epithelial-like morphology which remains constant during passaging (FIG. 6).

CEEK-TT1 cells of different passages have been frozen down in ampoules for liquid nitrogen storage. These cells could easily be regrown after removal from the liquid nitrogen storage.

CEEK-TT2

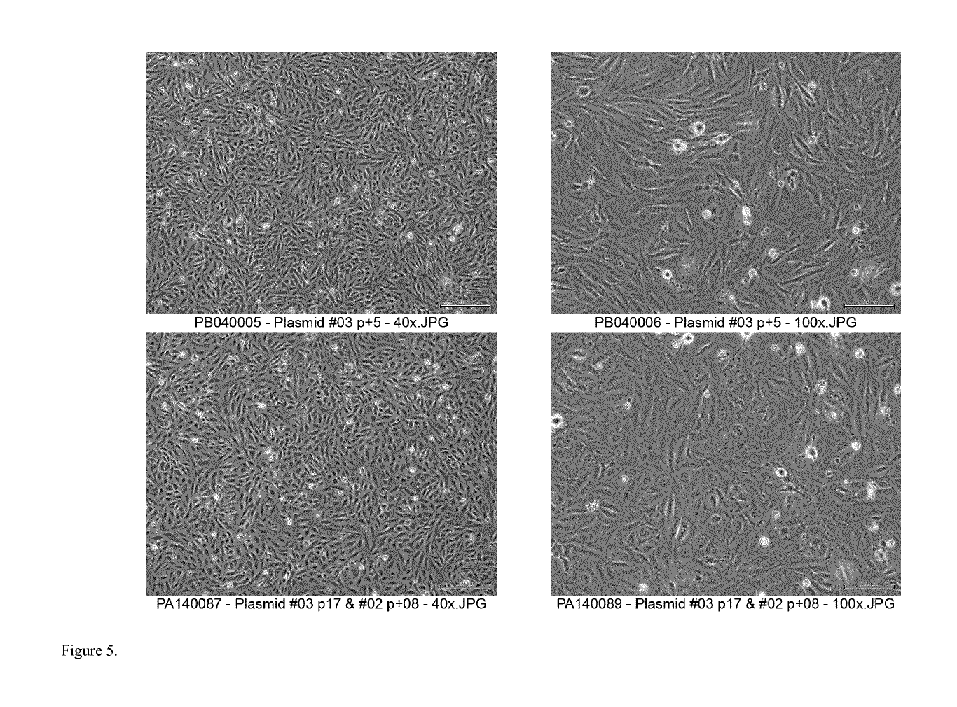

The observation that only CEEK cells that were co-transfected with plasmids #02 & #03 & #04 were immortalized (FIG. 4) indicates that expression of both SV40 T Ag and ChTERT are required for immortalization of CEEK cells. Therefore, cells that were initially transfected using only plasmids #03 & #04 were transfected a second time after culturing for 17 passages after initial transfection. The second transfection was performed with plasmids #02 & #04 in order to add stable expression of ChTERT to these cells. In contrast to the control (cells transfected using only plasmids #03 & #04) that went into crisis and died around passage 21, the twice transfected cells stably expressing SV40 T Ag and ChTERT continued to divide and proliferated well. These cells have been passaged for at least 17+21 passages and have performed at least 104 PDs. These cells continue to proliferate vigorously after passage 17+21 (104 PDs). The cells also have an epithelial-like morphology which remains constant during passaging (FIG. 5). Therefore, it is concluded that a second immortalized CEEK cell line was created, henceforward called CEEK-TT2 (Chicken Embryonic Epithelial Kidney cells—SV40 T Ag & ChTERT no. 2). CEEK-TT2 cells of different passages have been frozen down in ampoules for liquid nitrogen storage. These cells could easily be regrown after removal from the liquid nitrogen storage.

### REFERENCE LIST

Venkatesan, R. N. and Price, C. (1998). Telomerase expression in chickens: constitutive activity in somatic tissues and down-regulation in culture. Proc. Natl. Acad. Sci. U.S.A 95, 14763-14768.
Willemse, M. J., Strijdveen, I. G., van Schooneveld, S. H., van den Berg, M. C., and Sondermeijer, P. J. (1995). Transcriptional analysis of the short segment of the feline herpesvirus type 1 genome and insertional mutagenesis of a unique reading frame. Virology 208, 704-711.
Yusa, K., Rad, R., Takeda, J., and Bradley, A. (2009). Generation of transgene-free induced pluripotent mouse stem cells by the piggyBac transposon. Nat. Methods 6, 363-369.
Yusa, K., Zhou, L., Li, M. A., Bradley, A., and Craig, N. L. (2011). A hyperactive piggyBac transposase for mammalian applications. Proc. Natl. Acad. Sci. U.S.A 108, 1531-1536.
Sutterlin, G. G. and Laverty, G (1998). Characterization of a primary cell culture model of the avian renal proximal tubule. Am. J. Physiol. 275 (regulatory Integrative Comp. Physiol. 44): R220-R226.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A stably transfected immortalised chicken embryonic epithelial kidney cell (CEEK cell), wherein said immortalised CEEK cell comprises;
a) a gene encoding SV40 T antigen under the control of a suitable promoter;
b) a gene encoding chicken telomerase (cTERT) under the control of a suitable promoter; and
c) does not comprise exogenous retroviral Long Terminal Repeat (LTR) DNA.

15. A cell culture comprising immortalised CEEK cells of claim 14.

16. The cell culture of claim 15, wherein the cell culture is infected with an avian virus or avian viral vector.

17. The cell culture of claim 16, wherein the avian virus or avian viral vector is selected from the group consisting of Marek's Disease virus (MDV), the MDV-related Herpes virus of turkey (HVT), Newcastle Disease virus (NDV), Infectious Bronchitis virus (IBV), Infectious Bursal Disease virus (IBDV), Egg Drop Syndrome virus (EDSV), Turkey Rhinotracheitis virus (TRT), Reovirus (RV) and a HVT vector comprising an IBDV VP2-gene, an IBV-spike protein gene, an avian influenza HA gene, an ILT gD/gI protein gene or an NDV F-gene.

18. A method for the preparation of the immortalised CEEK cell of claim 14, wherein said method comprises;
a) obtaining primary CEEK cells;
b) transfecting said primary CEEK cells with;
1) a DNA molecule free of the LTR sequences, comprising transposon inverted repeats and comprising a gene encoding the SV40 T antigen under the control of a suitable promoter;
2) a DNA molecule free of the LTR sequences, comprising transposon inverted repeats and comprising a gene encoding chicken telomerase (cTERT) under the control of a suitable promoter; and
3) a DNA molecule comprising a gene encoding transposase under the control of a suitable promoter; and
c) selecting CEEK cells that have been cultured for at least 25 cell cycles.

19. The method for the preparation of the immortalised CEEK cell of claim 14, wherein said method comprises;
a) obtaining primary CEEK cells;
b) transfecting said primary CEEK cells with a single DNA molecule free of the LTR sequences, comprising transposon inverted repeats, comprising a gene encoding the SV40 T antigen under the control of a suitable promoter, comprising a gene encoding chicken telomerase under the control of a suitable promoter; and comprising a gene encoding transposase under the control of a suitable promoter; and
c) selecting CEEK cells that have been cultured for at least 25 cell cycles.

20. The method for the preparation of the immortalised CEEK cell of claim 14, wherein said method comprises;
a) obtaining primary CEEK cells;
b) transfecting said primary CEEK cells with;
1) a DNA molecule free of the LTR sequences, comprising transposon inverted repeats, and comprising a gene encoding the SV40 T antigen under the control of a suitable promoter and a gene encoding chicken telomerase under the control of a suitable promoter; and
2) a DNA molecule comprising a gene encoding transposase under the control of a suitable promoter;
c) selecting CEEK cells that have been cultured for at least 25 cell cycles.

21. The method of claim 18 wherein cells in step c) have been cultured for at least 50 cell cycles.

22. A method for the replication of an avian virus or avian viral vector, said method comprises;
a) culturing the immortalised CEEK cell of claim 14;
b) contacting the immortalised CEEK cell with the avian virus or avian viral vector; and
c) allowing the avian virus or avian viral vector to replicate.

23. The method of claim 22, wherein the avian virus or avian viral vector is selected from the group of avian viruses consisting of Marek's Disease virus (MDV), the MDV-related Herpes virus of turkey (HVT), Newcastle Disease virus (NDV), Infectious Bronchitis virus (IBV), Infectious Bursal Disease virus (IBDV), Egg Drop Syndrome virus (EDSV), Turkey Rhinotracheitis virus (TRT), Reovirus (RV) and a HVT vector comprising an IBDV VP2-gene, an IBV-spike protein gene, an avian influenza HA gene, an ILT gD/gI protein gene or an NDV F-gene.

24. A method for the preparation of a vaccine comprising an avian virus or an avian viral vector, wherein the method comprises the step of mixing a cell culture of claim 16 with a pharmaceutically acceptable carrier.

25. A vaccine comprising the cell culture of claim 16, and a pharmaceutically acceptable carrier.

26. A method for the preparation of a vaccine comprising an avian virus or an avian viral vector, wherein the method comprises the steps of;
a) infecting the cell culture of claim 14 with an avian virus or an avian viral vector;
b) replicating said avian virus or an avian viral vector;
c) isolating the progeny virus; and
d) mixing the progeny virus with a pharmaceutically acceptable carrier.
